(19) United States
(12) Patent Application Publication
(10) Pub. No.: US 2004/0192919 A1
(43) Pub. Date: Sep. 30, 2004

(54) Process for production of highly pure donepezil hydrochloride

(75) Inventors: Arie L. Gutman, Haifa (IL); Boris Tishin, Haifa (IL); Alexander Vilensky, Haifa (IL); Pavel Potyabin, Haifa (IL); Gennady A. Nisnevich, Haifa (IL)

Correspondence Address: STERNE, KESSLER, GOLDSTEIN & FOX PLLC, 1100 NEW YORK AVENUE, N.W., WASHINGTON, DC, 20005, US

(73) Assignee: FineTech Laboratories, Ltd.

(21) Appl. No.: 10/640,419

(22) Filed: Aug. 14, 2003

Related U.S. Application Data
(60) Provisional application No. 60/444,653, filed on Feb. 4, 2003.

(30) Foreign Application Priority Data
Aug. 14, 2002 (IL) 151253

(51) Int. Cl. C07D 211/06

(52) U.S. Cl. 546/205

(57) ABSTRACT

Disclosed is a process for production of highly pure donepezil hydrochloride that does not involve the isolation of donepezil base. The disclosed process involves intramolecular cyclization of 2-(3,4-dimethoxybenzyl)-3-(N-benzyl-4-piperidine)propionic acid followed by treatment with HCl.

### BACKGROUND OF THE INVENTION

1. Field of the Invention

The present invention relates to a process for the preparation of highly pure donepezil hydrochloride.

Related Art

Donepezil hydrochloride, a reversible acetylcholinesterase inhibitor represented by the following formula,

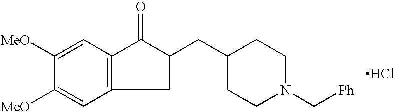

is an active ingredient marketed in the United States of America under the proprietary name ARICEPT (Eisai Medical Research). Donepezil hydrochloride is useful as a therapeutic and ameliorating agent for Alzheimer's-type senile dementia, particularly as a prophylactic treatment.

Donepezil hydrochloride is produced by first producing donepezil, which is a free base, and then converting it into a hydrochloride. In other words, donepezil base is used as a precursor for the production of donepezil hydrochloride.

A synthetic route to donepezil is shown in Scheme 1 and comprises the condensation of 5,6-dimethoxy-1-indanone with 1-benzyl-4-piperidinecarboxaldehyde followed by reduction of the obtained compound [III] and column chromatography of the crude donepezil base on silica gel. Donepezil hydrochloride is prepared from the resulting purified donepezil base by treating the base with HCl/EtOAc and re-crystallizing the resulting solid from MeOH/i-Pr₂O (H. Sugimoto et al., J. Med. Chem. 38:481 (1995); U.S. Pat. No. 5,100,901).

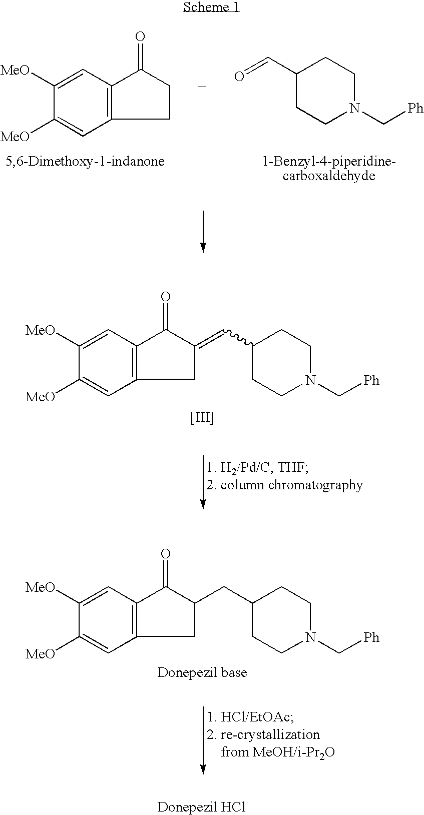

Recently it was proposed to use crystallization for purification of crude donepezil base obtained after the reduction of compound [III] (U.S. Pat. No. 6,245,911).

An alternative process disclosed in WO 00/09483 for preparing donepezil base is shown in Scheme 2 and comprises the steps of (a) carrying out the intramolecular cyclization of compound [II] to yield donepezil base and (b) isolating the resulting donepezil base by chromatography or crystallization.

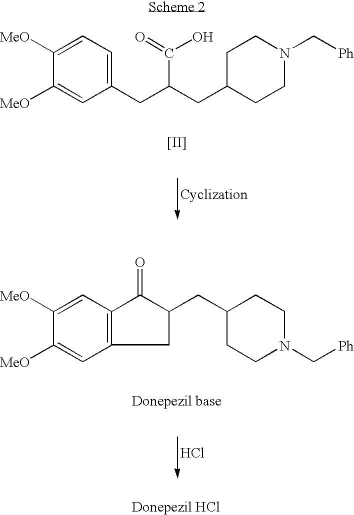

An additional process for production of donepezil hydrochloride involves reacting a donepezil intermediate with halogenated benzyl to obtain a donepezil quaternary ammonium salt, hydrogenation of the quaternary ammonium salt to produce donepezil base, followed by addition of HCl to produce donepezil hydrochloride (U.S. Pat. No. 6,252,081).

It should be noted that all of the known procedures for preparing donepezil hydrochloride involve the isolation and handling of intermediate donepezil base prior to converting it into donepezil hydrochloride.

There are several disadvantages in such two-step processes. These include increased cycle time due to increased handling requirements, increased air emissions if drying of the donepezil base is required, and increased total volume of solvent needed. Further, in the prior art, the desired compound is purified by column chromatography, which requires a large amount of solvent and the column is discarded after use, and is thus very disadvantageous in production cost. In addition, a large amount of used solvent is evaporated into the atmosphere. Therefore, column chromatography is preferably not used from the viewpoint of working environment and environmental protection.

### SUMMARY OF THE INVENTION

In view of the foregoing, there is a need in the art to provide a scalable industrial process for synthesizing highly pure donepezil hydrochloride, without the need for isolation, chromatography and re-crystallization of the intermediate donepezil base. It is a further need in the art to provide a process for synthesizing donepezil hydrochloride having a liquid chromatography (LC) purity (by area under a LC peak) of more than 97% and a content of each individual impurity not exceeding 0.02% (by area).

Thus, the present invention provides a process for preparing highly pure donepezil hydrochloride represented by the following formula

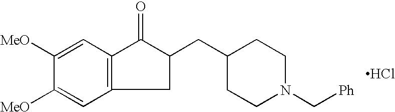

wherein the obtained salt has a liquid chromatography (LC) purity (by area) of more than 97% and a content of each individual impurity not exceeding 0.02% (by area), which process comprises:

carrying out an intramolecular cyclization of 2-(3,4-dimethoxybenzyl)-3-(N-benzyl-4-piperidine)propionic acid [II]

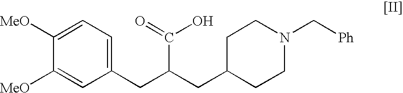

or its salt to form donepezil base; treating the donepezil base with HCl without isolating the donepezil base to form donepezil hydrochloride; and crystallizing the donepezil hydrochloride to give the salt of desired LC purity.

### DETAILED DESCRIPTION OF THE INVENTION

The described invention provides a process for the preparation of highly pure donepezil hydrochloride by cyclization of a compound of formula [II] or a salt thereof.

Preferably, cyclization of compound [II] is carried out under Friedel-Crafts reaction conditions, optionally with previous derivatization of the carboxylic group to a halocarbonyl group.

Preferably, the cyclization in the process of the present invention is carried out in the presence of protic acids or Lewis acids or a mixture of protic and Lewis acids. Examples of protic acids are trifluoromethanesulfonic acid, methanesulfonic acid, polyphosphoric acid, fluorosulfonic acid, chlorosulfonic acid, sulfuric acid, hydrogen fluoride, and hydrogen chloride. Examples of Lewis acids are zinc chloride, zinc bromide, aluminum chloride, aluminum bromide, titanium chloride, boron fluoride, phosphorus pentoxide, phosphorus oxychloride, phosphorus pentachloride, phosphorus trichloride, thionyl chloride, and sulfuryl chloride.

The cyclization may be carried out in the presence of a solvent. Preferably, the solvent is a halogenated solvent selected from dichloromethane, chloroform, dichloroethane, tetrachloroethane, chlorobenzene, dichlorobenzene, or may be nitromethane, nitroethane, nitrobenzene, ether and/or mixtures thereof.

After mixing compound [II] with a solvent and a protic acid and/or Lewis acid, the mixture is stirred at a temperature in the range of 0-100° C., preferably in the range of 10-40° C., most preferably in the range of 15-30° C. The cyclization process can be carried out for a time period of 1-10 hours, preferably 2-6 hours, more preferably 3-4 hours.

The carboxylic group of compound [II] can be derivatized to a halocarbonyl group using compounds such as phosphorus trichloride, thionyl chloride or oxalyl chloride. Smith et al., March's Advanced Organic Chemistry, 5^{th} Ed., Wiley, & Sons, Inc., New York, p. 523. The derivatization can be carried out prior to the cyclization process and the derivatized compound added to the cyclization reaction mix. Alternatively, the derivatization can be carried out in situ during the cyclization process.

After the cyclization process the reaction mix can be cooled by pouring the mix into ice. After cooling, the mix can optionally be basified to a pH of about 9-10, for example with potassium hydroxide or sodium carbonate. The organic layer is removed, dried over a dehydrating material, e.g., anhydrous sodium sulfate, and concentrated under reduced pressure. Optionally, the aqueous layer can be extracted with solvent, e.g., dichloromethane, and the organic extracts combined prior to drying and concentrating.

Preferably, the compound [II] or its salt has a liquid chromatography purity (by area) of at least 97%. More preferably, the compound [II] or its salt has a liquid chromatography purity (by area) of at least 98%. More preferably, the compound [II] or its salt has a liquid chromatography purity (by area) of at least 99%. Most preferably, the compound [II] or its salt has a liquid chromatography purity (by area) of at least 99.9%.

The following example is illustrative, but not limiting, of the methods of the present invention. Other suitable modifications and adaptations of the variety of conditions and parameters normally encountered in chemical synthesis and which are obvious to those skilled in the art are within the spirit and scope of the invention.

### EXAMPLE 1

### Highly Pure Donepezil Hydrochloride

The liquid chromatography (LC) purity was determined by separating a sample by high performance liquid chromatography (HPLC) under the following conditions, and calculating the area percentage thereof of each peak.

HPLC Conditions:

Column: Intersil 5 mm ODS-2 150×4.6 mm (METACHEM)

Mobile phase: acetonitrile/water/70% aq. perchloric acid 30:70:0.1 (v/v)

Flow rate: 0.7 mL/min

Column temperature: 36° C.

Detection: UV 230 nm

A 100 L glass reactor equipped with a mechanical stirrer, a dropping funnel, a thermometer and a condenser connected to a bubbler was charged with phosphorus pentoxide (1.53 Kg) and methanesulfonic acid (14.6 Kg) and filled with argon. The mixture was stirred at 70-80° C. until completely homogenized. Dichloromethane (12 L) and 2-(3,4-dimethoxybenzyl)-3-(N-benzyl-4-piperidine)propionic acid [II] (2.75 Kg) were added to the mixture at 15-30° C. The obtained mixture was stirred under reflux conditions for 3 hours. The cold reaction mixture was poured into ice (40 Kg). The mixture was basified to pH 9-10 with 40% aq. potassium hydroxide at 10-15° C. The organic layer was separated and the aqueous layer was extracted with dichloromethane. The combined organic extracts were dried over sodium sulfate and concentrated to a volume of 7-8 L. The concentrated solution was added dropwise to a stirred 10% (by weight) solution of hydrogen chloride in ethyl acetate (3.40 Kg) while maintaining the temperature at 0-3° C. The mixture was stirred for 1 hour at 0-15° C. and evaporated under reduced pressure to obtain crystals, which were re-crystallized from methanol/isopropyl ether to obtain 2.45 Kg (85%) of donepezil hydrochloride with an HPLC purity (by area) of more than 99.9% and a content of any individual impurity not exceeding 0.02% (by area).

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A process for the preparation of highly pure donepezil hydrochloride represented by the following formula 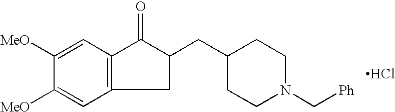
having a liquid chromatography (LC) purity of more than 97%, with a content of each individual impurity not exceeding 0.02%, which process comprises:
carrying out an intramolecular cyclization of 2-(3,4-dimethoxybenzyl)-3-(N-benzyl-4-piperidine)propionic acid [II] 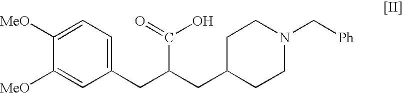
or its salt to form donepezil base; treating the donepezil base with HCl without isolating the donepezil base to form donepezil hydrochloride; and crystallizing the donepezil hydrochloride to give donepezil hydrochloride having a LC purity of more than 97% and a content of each individual impurity not exceeding 0.02%.

2. The process of claim 1, wherein the intramolecular cyclization is performed in the presence of a protic acid, a Lewis acid, or a mixture thereof.

3. The process of claim 2, wherein said intramolecular cyclization is performed in the presence of a protic acid selected from the group consisting of trifluoromethanesulfonic acid, methanesulfonic acid, polyphosphoric acid, fluorosulfonic acid, chlorosulfonic acid, sulfuric acid, hydrogen fluoride, and hydrogen chloride.

4. The process of claim 2, wherein said intramolecular cyclization is performed in the presence of a Lewis acid selected from the group consisting of zinc chloride, zinc bromide, aluminum chloride, aluminum bromide, titanium chloride, boron fluoride, phosphorus pentoxide, phosphorus oxychloride, phosphorus pentachloride, phosphorus trichloride, thionyl chloride, and sulfuryl chloride.

5. The process of claim 1, wherein said intramolecular cyclization is carried out in the present of a solvent.

6. The process of claim 5, wherein said solvent is a halogenated solvent.

7. The process of claim 6, wherein said halogenated solvent is selected from the group consisting of dichloromethane, chloroform, dichloroethane, tetrachloroethane, chlorobenzene, and dichlorobenzene and mixtures thereof.

8. The process of claim 5, wherein said solvent is selected from the group consisting of nitromethane, nitroethane, nitrobenzene, and ether and mixtures thereof.

9. The process of claim 1, wherein the carboxylic group of compound [II] is derivatized to a halocarbonyl group prior to carrying out the intramolecular cyclization.

10. The process of claim 1, wherein when said donepezil base is treated with HCl, a donepezil hydrochloride-containing solution is obtained, and said donepezil hydrochloride-containing solution is evaporated under reduced pressure to obtain donepezil hydrochloride crystals and said donepezil hydrochloride crystals are re-crystallized from methanol/isopropyl ether.

11. The process of claim 1, wherein the donepezil hydrochloride has a LC purity of more than 98%.

12. The process of claim 1, wherein the donepezil hydrochloride has a LC purity of more than 99%.

13. The process of claim 1, wherein the donepezil hydrochloride has a LC purity of more than 99.9%.

14. Donepezil hydrochloride having a LC purity of more than 99.9%, with a content of each individual impurity not exceeding 0.02%, prepared by the process of claim 1.
